(19) United States
(12) Patent Application Publication
(10) Pub. No.: US 2019/0192525 A1
(43) Pub. Date: Jun. 27, 2019

(54) APILIMOD COMPOSITIONS AND METHODS FOR USING SAME

(71) Applicant: Al Therapeutics, Inc., Guilford, CT (US)

(72) Inventors: Henri Lichenstein, Guilford, CT (US); Johnathan M. Rothberg, Guilford, CT (US)

(21) Appl. No.: 16/289,963

(22) Filed: Mar. 1, 2019

Related U.S. Application Data
(62) Division of application No. 15/548,222, filed on Aug. 2, 2017.
(60) Provisional application No. 62/111,476, filed on Feb. 3, 2015.

(51) Int. Cl. A61K 31/5377; A61K 45/06; A61K 9/00; A61K 31/519; A61K 31/404; A61K 31/439; A61K 31/4178; A61K 31/473

(52) U.S. Cl. CPC A61K 31/5377; A61K 45/06; A61K 9/0019; A61K 31/473; A61K 31/404; A61K 31/439; A61K 31/4178; A61K 31/519

(57) ABSTRACT

The present invention relates to methods for treating Charcot-Marie-Tooth Disease (CMT) with apilimod and related compositions and methods.

### RELATED APPLICATION

This application claims priority to U.S. Provisional Patent Application Ser. No. 62/111,476, filed on Feb. 3, 2015, the contents of which are hereby fully incorporated by reference.

### FIELD OF THE INVENTION

The present invention relates to compositions comprising apilimod and methods of using same to treat Charcot-Marie-Tooth disease (CMT).

### BACKGROUND OF THE INVENTION

Charcot-Marie-Tooth disease (CMT) is one of the most common inherited neurological disorders, affecting approximately 1 in 2,500 people in the United States. The disease is named for the three physicians who first identified it in 1886—Jean-Martin Charcot and Pierre Marie in Paris, France, and Howard Henry Tooth in Cambridge, England. CMT, also known as hereditary motor and sensory neuropathy (HMSN) or peroneal muscular atrophy, comprises a group of disorders that affect peripheral nerves. The peripheral nerves lie outside the brain and spinal cord and supply the muscles and sensory organs in the limbs.

CMT is caused by mutations in genes that produce proteins involved in the structure and function of either the peripheral nerve axon or the myelin sheath. Although different proteins are abnormal in different forms of CMT disease, all of the mutations affect the normal function of the peripheral nerves. Consequently, these nerves slowly degenerate and lose the ability to communicate with their distant targets. The degeneration of motor nerves results in muscle weakness and atrophy in the extremities (arms, legs, hands, or feet), and in some cases the degeneration of sensory nerves results in a reduced ability to feel heat, cold, and pain. The gene mutations in CMT disease are usually inherited. Individuals normally possess two copies of every gene, one inherited from each parent. Some forms of CMT are inherited in an autosomal dominant fashion while other forms of CMT are inherited in an autosomal recessive fashion. Additional forms of CMT are also inherited in an X-linked fashion, which means that the abnormal gene is located on the X chromosome. In some rare cases the gene mutation causing CMT disease is a new mutation which occurs spontaneously in the individual's genetic material and has not been passed down through the family.

Symptoms of CMT usually begin in late childhood or early adulthood, but can begin earlier. Some people do not experience symptoms until their early thirties or forties. Usually, the initial symptom is foot drop early in the course of the disease. This can also cause hammer toe, where the toes are always curled. Wasting of muscle tissue of the lower parts of the legs may give rise to a “stork leg” or “inverted champagne bottle” appearance. Weakness in the hands and forearms occurs in many people as the disease progresses.

Loss of touch sensation in the feet, ankles and legs, as well as in the hands, wrists and arms occur with various types of the disease. Early and late onset forms occur with ‘on and off’ painful spasmodic muscular contractions that can be disabling when the disease activates. High arched feet (pes cavus) or flat arched feet (pes planus) are classically associated with the disorder. Sensory and proprioceptive nerves in the hands and feet are often damaged, while pain nerves are left intact. Overuse of an affected hand or limb can activate symptoms including numbness, spasm, and painful cramping.

Symptoms and progression of the disease can vary. Breathing can be affected in some; so can hearing, vision, as well as the neck and shoulder muscles. Scoliosis is common. Hip sockets can be malformed. Gastrointestinal problems can be part of CMT, as can difficulty chewing, swallowing, and speaking (due to atrophy of vocal cords). A tremor can develop as muscles waste. Pregnancy has been known to exacerbate CMT, as well as extreme emotional stress. Patients with CMT must avoid periods of prolonged immobility such as when recovering from a secondary injury as prolonged periods of limited mobility can drastically accelerate symptoms of CMT.

CMT can be diagnosed based upon clinical symptoms, through measurement of the speed of nerve impulses (nerve conduction studies), through biopsy of the nerve, and through DNA testing. DNA testing can give a definitive diagnosis, but not all the genetic markers for CMT are known. CMT is first noticed when individuals develop lower leg weakness, such as foot drop; or foot deformities, including hammertoes and high arches. But signs alone do not lead to diagnosis. Patients must be referred to a physician specializing in neurology or rehabilitation medicine. The lack of family history does not rule out CMT, but it will allow the doctor to rule out other causes of neuropathy such as diabetes or exposure to certain chemicals or drugs.

There is no current standard treatment. Often the most important goal for patients with CMT is to maintain movement, muscle strength, and flexibility. Therefore, physical therapy and moderate activity are usually recommended, but overexertion should be avoided. A physiotherapist should be involved in designing an exercise program that fits a patient's personal strengths and flexibility. Orthoses (bracing) can also be used to correct problems caused by CMT. CMT patients must take extra care to avoid falling because fractures take longer to heal in someone with an underlying disease process. Additionally, the resulting inactivity may cause the CMT to worsen. Pain due to postural changes, skeletal deformations, muscular fatigue and cramping is fairly common in people with CMT. It can be mitigated or treated by physical therapies, surgeries, and corrective or assistive devices. Analgesic medications may also be needed if other therapies do not provide relief from pain. Neuropathic pain is often a symptom of CMT, though, like other symptoms of CMT, its presence and severity varies from case to case.

Although much research has been undertaken in this field, there are currently no effective treatment options available to patients beyond what is essentially palliative care. Current clinical trials within the United States are investigating substances like coenzyme Q, ascorbic acid and PXT3003, which have shown promise in animal models of neurological disorders. There remains a need for effective therapies for the treatment of CMT.

### SUMMARY OF THE INVENTION

The present invention relates to a new use of apilimod for treating CMT. The present invention is based in part on the surprising discovery that apilimod is a highly selective PIKfyve inhibitor. This activity was not predicted based upon apilimod's immunomodulatory activity via its known inhibition of IL-12/23 production. PIKfyve is a phosphoinositide kinase (PIK) that contains a FYVE-type zinc finger domain, which binds phosphatidylinositol 3-phosphate (PI3P). PIKfyve phosphorylates PI3P to produce PI(3,5)P₂, which is involved in cellular processes including membrane trafficking and cytoskeletal reorganization. A screen of over 450 kinases identified PIKfyve as the only high affinity binding target (Kd=75 pM) for apilimod in human cells.

Alterations in phosphoinositol (PI) signaling and vesicle trafficking have been implicated in CMT disease. Neurons seem to be particularly sensitive to the levels of PI(3,5)P₂, as evidenced by mutations in PI(3,5)P₂-related genes which are implicated in multiple neurological disorders. PI(3,5)P₂ also plays a role in controlling synapse function and/or plasticity. As noted above, PI(3,5)P₂ is generated from PI3P by PIKfyve. An imbalance of PI(3,5)P₂ in Schwann cells has been implicated in causing myelin outfoldings in MTMR2-null nerves. These myelin outfoldings in the nerves consist of redundant loops of myelin around a main myelinated axon and are a hallmark of CMT4B disorders. Genetic and pharmacological inhibition of PIKfyve rescues myelin outfoldings both in vitro and in vivo.

Accordingly, in one aspect, the present invention provides a method for treating CMT in a subject in need thereof, the method comprising administering to the subject a therapeutically effective amount of an apilimod composition of the invention, said composition comprising apilimod, or a pharmaceutically acceptable salt, solvate, clathrate, hydrate, polymorph, prodrug, analog or derivative thereof. In one embodiment, the apilimod composition comprises apilimod free base or apilimod dimesylate. In one embodiment, the CMT is a subtype selected from the group consisting of CMT1, CMT2, CMT3, CMT4, and CMTX. In one embodiment, the CMT is CMT4. In one embodiment, the method further comprises administering at least one additional active agent to the subject. The at least one additional active agent may be a therapeutic agent or a non-therapeutic agent. The at least one additional active agent may be administered in a single dosage form with the apilimod composition, or in a separate dosage form from the apilimod composition. In one embodiment, the at least one additional active agent is selected from the group consisting of an analgesic agent, a progesterone antagonist, a histone deacetylase inhibitor, a tricyclic antidepressant, anticonvulsant and combinations thereof. In one embodiment, the at least one additional active agent is a therapeutic agent selected from the group consisting of ibuprofen, acetaminophen, naproxen, onapristone, desipramine, doxepin, nortriptyline, amitriptyline, gabapentin, and combinations thereof. In one embodiment, the at least one additional active agent is a non-therapeutic agent selected to ameliorate one or more side effects of the apilimod composition. In one embodiment, the non-therapeutic agent is selected from the group consisting of ondansetron, granisetron, dolasetron and palonosetron. In one embodiment, the non-therapeutic agent is selected from the group consisting of pindolol and risperidone. In one embodiment, the at least one additional active agent is a non-therapeutic agent selected to ameliorate one or more symptoms CMT. In one embodiment, the non-therapeutic agent is selected from the group consisting of physico therapy, stem cell therapy, gene therapy, physiotherapy, and combinations thereof.

In one embodiment, the dosage form of the apilimod composition is an oral dosage form. In another embodiment, the dosage form of the apilimod composition is suitable for intravenous administration. In one embodiment, where the dosage form is suitable for intravenous administration, administration is by a single injection or by a drip bag.

In one embodiment, the subject is a human CMT patient. In one embodiment, the human CMT patient in need of treatment with an apilimod composition of the invention is one who has been diagnosed with CMT or presents with one or more CMT-related symptoms.

In one embodiment, the method is a method of treating CMT using a combination therapy comprising an apilimod composition and an analgesic agent for the treatment of the CMT.

The invention also provides methods of reducing PI(3,5)P₂ levels in neuronal cells, the method comprising delivering to the cells an apilimod composition in an amount effective to selectively inhibit PIKfyve activity in the neuronal cells.

### DETAILED DESCRIPTION OF THE INVENTION

The present invention provides compositions and methods related to the use of apilimod for treating and/or preventing CMT in a subject, preferably a human subject, in need of such treatment. The present invention further provides compositions and methods related to the use of apilimod for maintaining CMT in a subject, preferably a human subject, in need thereof. In addition, the present invention also provides novel therapeutic approaches to CMT treatment based upon combination therapy utilizing apilimod and at least one additional therapeutic agent. The combination therapies described herein exploit the unique inhibitory activity of apilimod which may provide a synergistic effect when combined with other therapeutic agents.

As used herein, the term “an apilimod composition” may refer to a composition comprising apilimod itself (free base), or may encompass pharmaceutically acceptable salts, solvates, clathrates, hydrates, polymorphs, prodrugs, analogs or derivatives of apilimod as described below. The stmnctire of apilimod is shown in Formula I:

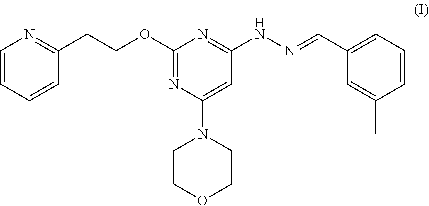 [SMILES: Cc1cccc(/C=N/Nc2cc(N3CCOCC3)nc(OCCc3ccccn3)n2)c1]

The chemical name of apilimod is 2-[2-Pyridin-2-yl)-ethoxy]-4-N′-(3-methyl-benzilidene)-hydrazino]-6-(morpholin-4-yl)-pyrimidine (IUPAC name: (E)-4-(6-(2-(3-methylbenzylidene)hydrazinyl)-2-(2-(pyridin-2-yl)ethoxy)pyrimidin-4-yl)morpholine), and the CAS number is 541550-19-0.

Apilimod can be prepared, for example, according to the methods described in U.S. Pat. Nos. 7,923,557, and 7,863,270, and WO 2006/128129.

As used herein, the term “pharmaceutically acceptable salt,” is a salt formed from, for example, an acid and a basic group of an apilimod composition. Illustrative salts include, but are not limited, to sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, besylate, gentisinate, fumarate, gluconate, glucaronate, saccharate, formate, benzoate, glutamate, methanesulfonate, ethanesulfonate, benzenesulfonate, p-toluenesulfonate, and pamoate (e.g., 1,1′-methylene-bis-(2-hydroxy-3-naphthoate)) salts. In a preferred embodiment, the salt of apilimod comprises methanesulfonate.

The term “pharmaceutically acceptable salt” also refers to a salt prepared from an apilimod composition having an acidic functional group, such as a carboxylic acid functional group, and a pharmaceutically acceptable inorganic or organic base.

The term “pharmaceutically acceptable salt” also refers to a salt prepared from an apilimod composition having a basic functional group, such as an amino functional group, and a pharmaceutically acceptable inorganic or organic acid.

The salts of the compounds described herein can be synthesized from the parent compound by conventional chemical methods such as methods described in Pharmaceutical Salts: Properties, Selection, and Use, P. Hemrich Stalil (Editor), Camille G. Wermuth (Editor), ISBN: 3-90639-026-8, August 2002. Generally, such salts can be prepared by reacting the parent compound with the appropriate acid in water or in an organic solvent, or in a mixture of the two.

One salt form of a compound described herein can be converted to the free base and optionally to another salt form by methods well known to the skilled person. For example, the free base can be formed by passing the salt solution through a column containing an amine stationary phase (e.g. a Strata-NH₂ column). Alternatively, a solution of the salt in water can be treated with sodium bicarbonate to decompose the salt and precipitate out the free base. The free base may then be combined with another acid using routine method.

As used herein, the term “polymorph” means solid crystalline forms of a compound of the present invention (e.g., 2-[2-Pyridin-2-yl)-ethoxy]-4-N′-(3-methyl-benzilidene)-hydrazino]-6-(morpholin-4-yl)-pyrimidine) or complex thereof. Different polymorphs of the same compound can exhibit different physical, chemical and/or spectroscopic properties. Different physical properties include, but are not limited to stability (e.g., to heat or light), compressibility and density (important in formulation and product manufacturing), and dissolution rates (which can affect bioavailability). Differences in stability can result from changes in chemical reactivity (e.g., differential oxidation, such that a dosage form discolors more rapidly when comprised of one polymorph than when comprised of another polymorph) or mechanical characteristics (e.g., tablets crumble on storage as a kinetically favored polymorph converts to thermodynamically more stable polymorph) or both (e.g., tablets of one polymorph are more susceptible to breakdown at high humidity). Different physical properties of polymorphs can affect their processing. For example, one polymorph might be more likely to form solvates or might be more difficult to filter or wash free of impurities than another due to, for example, the shape or size distribution of particles of it.

As used herein, the term “hydrate” means a compound of the present invention (e.g., 2-[2-Pyridin-2-yl)-ethoxy]-4-N′-(3-methyl-benzilidene)-hydrazino]-6-(morpholin-4-yl)-pyrimidine) or a salt thereof, which further includes a stoichiometric or non-stoichiometric amount of water bound by non-covalent intermolecular forces.

As used herein, the term “clathrate” means a compound of the present invention (e.g., 2-[2-Pyridin-2-yl)-ethoxy]-4-N′-(3-methyl-benzilidene)-hydrazino]-6-(morpholin-4-yl)-pyrimidine) or a salt thereof in the form of a crystal lattice that contains spaces (e.g., channels) that have a guest molecule (e.g., a solvent or water) trapped within.

As used herein, the term “prodrug” means a derivative of a compound described herein (e.g., 2-[2-Pyridin-2-yl)-ethoxy]-4-N′-(3-methyl-benzilidene)-hydrazino]-6-(morpholin-4-yl)-pyrimidine) that can hydrolyze, oxidize, or otherwise react under biological conditions (in vitro or in vivo) to provide a compound of the invention. Prodrugs may only become active upon such reaction under biological conditions, or they may have activity in their unreacted forms. Examples of prodrugs contemplated in this invention include, but are not limited to, analogs or derivatives of a compound described herein (e.g., 2-[2-Pyridin-2-yl)-ethoxy]-4-N′-(3-methyl-benzilidene)-hydrazino]-6-(morpholin-4-yl)-pyrimidine) that comprise biohydrolyzable moieties such as biohydrolyzable amides, biohydrolyzable esters, biohydrolyzable carbamates, biohydrolyzable carbonates, biohydrolyzable ureides, and biohydrolyzable phosphate analogues. Other examples of prodrugs include derivatives of compounds of any one of the formulae disclosed herein that comprise —NO, —NO₂, —ONO, or —ONO₂ moieties. Prodrugs can typically be prepared using well-known methods, such as those described by Burger's Medicinal Chemistry and Drug Discovery (1995) 172-178, 949-982 (Manfred E. Wolff ed., 5th ed).

As used herein, the term “solvate” or “pharmaceutically acceptable solvate,” is a solvate formed from the association of one or more solvent molecules to one of the compounds disclosed herein (e.g., 2-[2-Pyridin-2-yl)-ethoxy]-4-N′-(3-methyl-benzilidene)-hydrazino]-6-(morpholin-4-yl)-pyrimidine). The term solvate includes hydrates (e.g., hemi-hydrate, mono-hydrate, dihydrate, trihydrate, tetrahydrate, and the like).

As used herein, the term “analog” refers to a chemical compound that is structurally similar to another but differs slightly in composition (as in the replacement of one atom by an atom of a different element or in the presence of a particular functional group, or the replacement of one functional group by another functional group). Thus, an analog is a compound that is similar or comparable in function and appearance, but not in structure or origin to the reference compound. As used herein, the term “derivative” refers to compounds that have a common core structure, and are substituted with various groups as described herein.

### Charcot-Marie Tooth Disease

Charcot-Marie-Tooth disease (CMT), also known as Charcot-Marie-Tooth neuropathy, hereditary motor and sensory neuropathy (HMSN) and peroneal muscular atrophy (PMA), is a genetically and clinically heterogeneous group of inherited disorders of the peripheral nervous system characterized by progressive loss of muscle tissue and touch sensation across various parts of the body.

There are many forms and subtypes of CMT disease, including CMT1, CMT2, CMT3, CMT4, and CMTX. CMT1, caused by abnormalities in the myelin sheath.

For example: CMT1A is an autosomal dominant disease that results from a duplication of the gene on chromosome 17 that carries the instructions for producing the peripheral myelin protein-22 (PMP-22). The PMP-22 protein is a critical component of the myelin sheath. Overexpression of this gene causes the structure and function of the myelin sheath to be abnormal. Patients experience weakness and atrophy of the muscles of the lower legs beginning in adolescence; later they experience hand weakness and sensory loss. Interestingly, a different neuropathy distinct from CMT1A called hereditary neuropathy with predisposition to pressure palsy (HNPP) is caused by a deletion of one of the PMP-22 genes. In this case, abnormally low levels of the PMP-22 gene result in episodic, recurrent demyelinating neuropathy. CMT1B is an autosomal dominant disease caused by mutations in the gene that carries the instructions for manufacturing the myelin protein zero (P0), which is another critical component of the myelin sheath. Most of these mutations are point mutations, meaning a mistake occurs in only one letter of the DNA genetic code. To date, scientists have identified more than 120 different point mutations in the P0 gene. As a result of abnormalities in P0, CMT1B produces symptoms similar to those found in CMT1A. The less common CMT1C, CMT1D, and CMT1E, which also have symptoms similar to those found in CMT1A, are caused by mutations in the LITAF, EGR2, and NEFL genes, respectively.

CMT2 results from abnormalities in the axon of the peripheral nerve cell rather than the myelin sheath. It is less common than CMT1. CMT2A, the most common axonal form of CMT, is caused by mutations in Mitofusin 2, a protein associated with mitochondrial fusion. CMT2A has also been linked to mutations in the gene that codes for the kinesin family member 1B-beta protein, but this has not been replicated in other cases. Kinesins are proteins that act as motors to help power the transport of materials along the cell. Other less common forms of CMT2 have been recently identified and are associated with various genes: CMT2B (associated with RAB7), CMT2D (GARS). CMT2E (NEFL), CMT2H (HSP27), and CMT21 (HSP22). CMT3 or Dejerine-Sottas disease is a severe demyelinating neuropathy that begins in infancy. Infants have severe muscle atrophy, weakness, and sensory problems. This rare disorder can be caused by a specific point mutation in the P0 gene or a point mutation in the PMP-22 gene.

CMT4 comprises several different subtypes of autosomal recessive demyelinating motor and sensory neuropathies. Each neuropathy subtype is caused by a different genetic mutation, may affect a particular ethnic population, and produces distinct physiologic or clinical characteristics. Individuals with CMT4 generally develop symptoms of leg weakness in childhood and by adolescence they may not be able to walk. Several genes have been identified as causing CMT4, including GDAP1 (CMT4A), MTMR13 (CMT4B1), MTMR2 (CMT4B2), SH3TC2 (CMT4C), NDG1 (CMT4D), EGR2 (CMT4E), PRX (CMT4F), FDG4 (CMT4H), and FIG4 (CMT4J).

CMTX is caused by a point mutation in the connexin-32 gene on the X chromosome. The connexin-32 protein is expressed in Schwann cells-cells that wrap around nerve axons, making up a single segment of the myelin sheath. This protein may be involved in Schwann cell communication with the axon. Males who inherit one mutated gene from their mothers show moderate to severe symptoms of the disease beginning in late childhood or adolescence (the Y chromosome that males inherit from their fathers does not have the connexin-32 gene). Females who inherit one mutated gene from one parent and one normal gene from the other parent may develop mild symptoms in adolescence or later or may not develop symptoms of the disease at all. Charcot-Marie-Tooth disease is caused by mutations that cause defects in neuronal proteins. Nerve signals are conducted by an axon with a myelin sheath wrapped around it. Most mutations in CMT affect the myelin sheath, but some affect the axon.

The most common cause of CMT (70-80% of the cases) is the duplication of a large region on the short arm of chromosome 17 that includes the gene PMP22. Some mutations affect the gene MFN2, which codes for a mitochondrial protein. Cells contain separate sets of genes in their nucleus and in their mitochondria. In nerve cells, the mitochondria travel down the long axons. In some forms of CMT, mutated MFN2 causes the mitochondria to form large clusters, or clots, which are unable to travel down the axon towards the synapses. This prevents the synapses from functioning.

There is no cure for CMT and treatment typically involves palliative care including, for example, physical therapy, occupational therapy, braces and other orthopedic devices, orthopedic surgery, and analgesics.

### Methods of Treatment

The present invention provides methods for the treatment and/or prevention of CMT in a subject in need thereof by administering to the subject a therapeutically effective amount of an apilimod composition of the invention, said composition comprising apilimod, or a pharmaceutically acceptable salt, solvate, clathrate, hydrate, polymorph, prodrug, analog or derivative thereof. In one embodiment, the apilimod composition comprises apilimod free base or apilimod dimesylate. The present invention also provides methods for maintaining CMT in a subject in need thereof by administering to the subject a therapeutically effective amount of an apilimod composition of the invention, said composition comprising apilimod, or a pharmaceutically acceptable salt, solvate, clathrate, hydrate, polymorph, prodrug, analog or derivative thereof. In this context, the term “maintaining” refers to preventing the further progression of CMT in the subject. Where a subject is successfully maintained, in addition to slowing or preventing further disease progression, one or more symptoms associated with CMT may be reduced. In one embodiment, the apilimod composition comprises apilimod free base or apilimod dimesylate. The present invention further provides the use of an apilimod composition for the preparation of a medicament useful for the treatment of CMT.

In one embodiment, the CMT is selected from subtype CMT1 (CMT1A, CMT1B) CMT2, CMT3, CMT4 (CMT4J), CMT1C, CMT1D, CMT1E or CMTX. In one embodiment, the CMT is subtype CMT4. In one embodiment, the subtype is CMT4B.

### Combination Therapy

The present invention also provides methods comprising combination therapy. As used herein, “combination therapy” or “co-therapy” includes the administration of a therapeutically effective amount of an apilimod composition with at least one additional active agent, as part of a specific treatment regimen intended to provide a beneficial effect from the co-action of the apilimod composition and the additional active agent. “Combination therapy” is not intended to encompass the administration of two or more therapeutic compounds as part of separate monotherapy regimens that incidentally and arbitrarily result in a beneficial effect that was not intended or predicted.

In one embodiment, the method is a method of treating and/or preventing CMT using a combination therapy comprising an apilimod composition and analgesic agent. In one embodiment the analgesic agent comprises a nonsteroidal anti-inflammatory drug (NSAIDS), for example ibuprofen, acetaminophen, and naproxen. In another embodiment the analgesic agent comprises a COX-2 inhibitor, for example celecoxib.

Another aspect of the invention is a method of treating and/or preventing CMT using a combination therapy comprising an apilimod composition and a progesterone antagonist, for example onapristone.

Another aspect of the invention is a method of treating and/or preventing CMT using a combination therapy comprising an apilimod composition and a histone deacetylase (HDAC6) inhibitor.

Another aspect of the invention is a method of treating and/or preventing CMT using a combination therapy comprising an apilimod composition and tricyclic antidepressants, for example, desipramine, doxepin, nortriptyline, amitriptyline.

Another aspect of the invention is a method of treating and/or preventing CMT using a combination therapy comprising an apilimod composition and anticonvulsants, for example gabapentin.

The at least one additional active agent may be a therapeutic agent, for example an analgesic agent or an anticonvulsant agent, or a non-therapeutic agent, and combinations thereof. With respect to therapeutic agents, the beneficial effect of the combination includes, but is not limited to, pharmacokinetic or pharmacodynamic co-action resulting from the combination of therapeutically active compounds. With respect to non-therapeutic agents, the beneficial effect of the combination may relate to the mitigation of toxicity, side effect, or adverse event associated with a therapeutically active agent in the combination.

In one embodiment, the at least one additional agent is a non-therapeutic agent which mitigates one or more side effects of an apilimod composition, the one or more side effects selected from any of nausea, vomiting, headache, dizziness, lightheadedness, drowsiness and stress. In one aspect of this embodiment, the non-therapeutic agent is an antagonist of a serotonin receptor, also known as 5-hydroxytryptamine receptors or 5-HT receptors. In one aspect, the non-therapeutic agent is an antagonist of a 5-HT₃ or 5-HT₁ₐ receptor. In one aspect, the non-therapeutic agent is selected from the group consisting of ondansetron, granisetron, dolasetron and palonosetron. In another aspect, the non-therapeutic agent is selected from the group consisting of pindolol and risperidone. In another aspect, the non-therapeutic agent is selected from gene therapy, stem cell therapy, physiotherapy, physical therapy, foot care (e.g, custom-made shoes, leg braces, joint braces

In one embodiment, the at least one additional agent is a therapeutic agent. In one embodiment, the therapeutic agent is an NSAID agent. In one embodiment, the NSAID agent is naproxen. In one embodiment, an apilimod composition is administered along with naproxen in a single dosage form or in separate dosage forms. In one embodiment, the dosage form is an oral dosage form. In another embodiment, the dosage form is suitable for intravenous administration.

In the context of combination therapy, administration of the apilimod composition may be simultaneous with or sequential to the administration of the one or more additional active agents. In another embodiment, administration of the different components of a combination therapy may be at different frequencies. The one or more additional agents may be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a compound of the present invention.

The one or more additional active agents can be formulated for co-administration with an apilimod composition in a single dosage form, as described in greater detail herein. The one or more additional active agents can be administered separately from the dosage form that comprises the compound of the present invention. When the additional active agent is administered separately from the apilimod composition, it can be by the same or a different route of administration as the apilimod composition.

Preferably, the administration of an apilimod composition in combination with one or more additional agents provides a synergistic response in the subject being treated. In this context, the term “synergistic” refers to the efficacy of the combination being more effective than the additive effects of either single therapy alone. The synergistic effect of a combination therapy according to the invention can permit the use of lower dosages and/or less frequent administration of at least one agent in the combination compared to its dose and/or frequency outside of the combination. Additional beneficial effects of the combination can be manifested in the avoidance or reduction of adverse or unwanted side effects associated with the use of either therapy in the combination alone (also referred to as monotherapy).

“Combination therapy” also embraces the administration of the compounds of the present invention in further combination with non-drug therapies (e.g., surgery or physical therapy). Where the combination therapy further comprises a non-drug treatment, the non-drug treatment may be conducted at any suitable time so long as a beneficial effect from the co-action of the combination of the therapeutic compounds and non-drug treatment is achieved. For example, in appropriate cases, the beneficial effect is still achieved when the non-drug treatment is temporally removed from the administration of the therapeutic compounds, perhaps by days or even weeks.

The non-drug treatment can be selected from hormonal therapy, stem cell therapy, gene therapy, physical therapy, physiotherapy, foot care (e.g., customized shoes, leg braces, joint braces), and surgery.

In the context of the methods described herein, the amount of an apilimod composition administered to the subject is a therapeutically effective amount. The term “therapeutically effective amount” refers to an amount sufficient to treat, ameliorate a symptom of, reduce the severity of, or reduce the duration of the disease or disorder being treated, or enhance or improve the therapeutic effect of another therapy, or sufficient to exhibit a detectable therapeutic effect in the subject. In one embodiment, the therapeutically effective amount of an apilimod composition is the amount effective to inhibit PIKfyve kinase activity.

An effective amount of an apilimod composition can range from about 0.001 mg/kg to about 1000 mg/kg, about 0.01 mg/kg to about 100 mg/kg, about 10 mg/kg to about 250 mg/kg, about 0.1 mg/kg to about 15 mg/kg; or any range in which the low end of the range is any amount between 0.001 mg/kg and 900 mg/kg and the upper end of the range is any amount between 0.1 mg/kg and 1000 mg/kg (e.g., 0.005 mg/kg and 200 mg/kg, 0.5 mg/kg and 20 mg/kg). Effective doses will also vary, as recognized by those skilled in the art, depending on the diseases treated, route of administration, excipient usage, and the possibility of co-usage with other therapeutic treatments such as use of other agents. See, e.g., U.S. Pat. No. 7,863,270, incorporated herein by reference.

In more specific aspects, an apilimod composition is administered at a dosage regimen of 30-1000 mg/day (e.g., 30, 35, 40, 45, 50, 55, 60, 65, 70, 75, 80, 85, 90, 95, 100, 125, 150, 175, 200, 225, 250, 275, or 300 mg/day) for at least 1 week (e.g., 1, 2, 3, 4, 5, 6, 7, 8, 9, 10, 11, 12, 36, 48, or more weeks). Preferably, an apilimod composition is administered at a dosage regimen of 100-1000 mg/day for 4 or 16 weeks. Alternatively or subsequently, an apilimod composition is administered at a dosage regimen of 100 mg-300 mg twice a day for 8 weeks, or optionally, for 52 weeks. Alternatively or subsequently, an apilimod composition is administered at a dosage regimen of 50 mg-1000 mg twice a day for 8 weeks, or optionally, for 52 weeks.

An effective amount of the apilimod composition can be administered once daily, from two to five times daily, up to two times or up to three times daily, or up to eight times daily. In one embodiment, the apilimod composition is administered thrice daily, twice daily, once daily, fourteen days on (four times daily, thrice daily or twice daily, or once daily) and 7 days off in a 3-week cycle, up to five or seven days on (four times daily, thrice daily or twice daily, or once daily) and 14-16 days off in 3 week cycle, or once every two days, or once a week, or once every 2 weeks, or once every 3 weeks.

In accordance with the methods described herein, a “subject in need of” is a subject having a disease, disorder or condition, or a subject having an increased risk of developing a disease, disorder or condition relative to the population at large. The subject in need thereof can be one that is “non-responsive” to a currently available therapy for the disease or disorder, for example CMT. In this context, the terms “non-responsive” refers to the subject's response to therapy as not clinically adequate to relieve one or more symptoms associated with the disease or disorder. In one aspect of the methods described here, the subject in need thereof is a subject having CMT whose CMT is non-responsive to standard therapy.

A “subject” includes a mammal. The mammal can be e.g., any mammal, e.g., a human, primate, vertebrate, bird, mouse, rat, fowl, dog, cat, cow, horse, goat, camel, sheep or a pig. Preferably, the mammal is a human. The term “patient” refers to a human subject.

The present invention also provides a monotherapy for the treatment of a disease, disorder or condition as described herein. As used herein, “monotherapy” refers to the administration of a single active or therapeutic compound to a subject in need thereof.

As used herein, “treatment”, “treating” or “treat” describes the management and care of a patient for the purpose of combating a disease, condition, or disorder and includes the administration of an apilimod composition to alleviate the symptoms or complications of a disease, condition or disorder, or to eliminate the disease, condition or disorder.

As used herein, “prevention”, “preventing” or “prevent” describes reducing or eliminating the onset of the symptoms or complications of the disease, condition or disorder and includes the administration of an apilimod composition to reduce the onset, development or recurrence of symptoms of the disease, condition or disorder.

In one embodiment, the administration of an apilimod composition leads to the elimination of a symptom or complication of the disease or disorder being treated; however, elimination is not required. In one embodiment, the severity of the symptom is decreased. In the context of cancer, such symptoms may include clinical markers of severity or progression including the degree to which a tumor secrets growth factors, degrades the extracellular matrix, becomes vascularized, loses adhesion to juxtaposed tissues, or metastasizes, as well as the number of metastases.

Treating CMT according to the methods described herein can result in a reduction in pain intensity. Preferably, after treatment, the amount of pain experienced is reduced by 5% or greater relative to its pain intensity prior to treatment; more preferably, pain intensity is reduced by 10% or greater; more preferably, reduced by 20% or greater; more preferably, reduced by 30% or greater; more preferably, reduced by 40% or greater; even more preferably, reduced by 50% or greater; and most preferably, reduced by greater than 75% or greater. Pain intensity may be measured by any reproducible means of measurement. The pain intensity may be measured according to a pain scale.

Treating CMT according to the methods described herein can result in a reduction in nerve damage. Preferably, after treatment, pain intensity is reduced by 5% or greater relative to its degree of prior nerve damage; more preferably, nerve damage is reduced by 10% or greater; more preferably, reduced by 20% or greater; more preferably, reduced by 30% or greater; more preferably, reduced by 40% or greater; even more preferably, reduced by 50% or greater; and most preferably, reduced by greater than 75% or greater. Nerve damage may be measured by any reproducible means of measurement, for example using electrodiagnostic tests or nerve biopsy.

Treating a disorder, disease or condition according to the methods described herein can result in an increase in the quality of life of a population of treated subjects in comparison to a population receiving carrier alone. Preferably, the quality of life is increased significantly due to the reduction of negative symptoms. Negative symptoms may be reduced by 10% or greater; reduced by 20% or greater; reduced by 30% or greater; more preferably, reduced by 40% or greater; even more preferably, reduced by 50% or greater; and most preferably, reduced by greater than 75%.

Treating a disorder, disease or condition according to the methods described herein can result in a decrease of negative symptoms of a population of treated subjects in comparison to a population receiving monotherapy with a drug that is not an apilimod composition as described herein. Preferably, the decrease of negative symptoms of a population of treated subjects is reduced by 10% or greater; reduced by 20% or greater; reduced by 30% or greater; more preferably, reduced by 40% or greater; even more preferably, reduced by 50% or greater; and most preferably, reduced by greater than 75%. An increase in average survival time of a population may be measured by any reproducible means. A decrease of negative symptoms of a population may be measured, for example, by measuring for a population the degree of pain intensity or nerve damage compared to an untreated population.

Treating a disorder, disease or condition according to the methods described herein can result in a decrease in the rate of nerve damage. Preferably, after treatment, the rate of nerve damage is reduced by at least 5% relative to the rate of nerve damage prior to treatment; more preferably, the rate of nerve damage is reduced by at least 10%; more preferably, reduced by at least 20%; more preferably, reduced by at least 30%; more preferably, reduced by at least 40%; more preferably, reduced by at least 50%; even more preferably, reduced by at least 50%; and most preferably, reduced by at least 75%. The rate of nerve damage may be measured by any reproducible means of measurement, for example nerve biopsy, electrodiagnostic tests.

As used herein, the term “selectively” means tending to occur at a higher frequency in one population than in another population. The compared populations can be cell populations. Preferably, an apilimod composition as described herein acts selectively on. As used herein, a “normal cell” is a cell that cannot be classified as part of a “cell proliferative disorder”. A normal cell lacks unregulated or abnormal growth, or both, that can lead to the development of an unwanted condition or disease. Preferably, a normal cell possesses normally functioning cell cycle checkpoint control mechanisms. Preferably, an apilimod composition acts selectively to modulate one molecular target (e.g., a target kinase) but does not significantly modulate another molecular target (e.g., a non-target kinase). The invention also provides a method for selectively inhibiting the activity of an enzyme, such as a kinase. Preferably, an event occurs selectively in population A relative to population B if it occurs greater than two times more frequently in population A as compared to population B. An event occurs selectively if it occurs greater than five times more frequently in population A. An event occurs selectively if it occurs greater than ten times more frequently in population A; more preferably, greater than fifty times; even more preferably, greater than 100 times; and most preferably, greater than 1000 times more frequently in population A as compared to population B. For example, cell death would be said to occur selectively in diseased or hyper-proliferating cells if it occurred greater than twice as frequently in diseased or hyper-proliferating cells as compared to normal cells.

### Pharmaceutical Compositions and Formulations

The present invention provides apilimod compositions that are preferably pharmaceutically acceptable compositions suitable for use in a mammal, preferably a human. In this context, the compositions may further comprise at least one pharmaceutically acceptable excipient or carrier, wherein the amount is effective for the treatment of a disease or disorder. In one embodiment, the disease or disorder is cancer, preferably a lymphoma, and most preferably a B cell lymphoma. In one embodiment, the disease or disorder is an mTOR disease or disorder.

In one embodiment, the apilimod composition comprises apilimod free base or apilimod dimesylate.

In one embodiment, the apilimod composition is combined with at least one additional active agent in a single dosage form. In one embodiment, the composition further comprises an antioxidant.

In one embodiment, the at least one additional active agent is selected from the group consisting of an analgesic agent, an anticonvulsant agent, a progesterone antagonist, HDAC6 inhibitor, and antidepressant agent, and combinations thereof. In one embodiment, the at least one additional active agent is a therapeutic agent selected from the group consisting of Naproxen, celecoxib, gabapentin, and acetaminophen.

A “pharmaceutical composition” is a formulation containing the compounds described herein in a pharmaceutically acceptable form suitable for administration to a subject. As used herein, the phrase “pharmaceutically acceptable” refers to those compounds, materials, compositions, carriers, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

“Pharmaceutically acceptable excipient” means an excipient that is useful in preparing a pharmaceutical composition that is generally safe, non-toxic and neither biologically nor otherwise undesirable, and includes excipient that is acceptable for veterinary use as well as human pharmaceutical use. Examples of pharmaceutically acceptable excipients include, without limitation, sterile liquids, water, buffered saline, ethanol, polyol (for example, glycerol, propylene glycol, liquid polyethylene glycol and the like), oils, detergents, suspending agents, carbohydrates (e.g., glucose, lactose, sucrose or dextran), antioxidants (e.g., ascorbic acid or glutathione), chelating agents, low molecular weight proteins, or suitable mixtures thereof.

A pharmaceutical composition can be provided in bulk or in dosage unit form. It is especially advantageous to formulate pharmaceutical compositions in dosage unit form for ease of administration and uniformity of dosage. The term “dosage unit form” as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved. A dosage unit form can be an ampoule, a vial, a suppository, a dragee, a tablet, a capsule, an IV bag, or a single pump on an aerosol inhaler.

In therapeutic applications, the dosages vary depending on the agent, the age, weight, and clinical condition of the recipient patient, and the experience and judgment of the clinician or practitioner administering the therapy, among other factors affecting the selected dosage. Generally, the dose should be a therapeutically effective amount. Dosages can be provided in mg/kg/day units of measurement (which dose may be adjusted for the patient's weight in kg, body surface area in m², and age in years). An effective amount of a pharmaceutical composition is that which provides an objectively identifiable improvement as noted by the clinician or other qualified observer. For example, alleviating a symptom of a disorder, disease or condition. As used herein, the term “dosage effective manner” refers to amount of a pharmaceutical composition to produce the desired biological effect in a subject or cell.

For example, the dosage unit form can comprise 1 nanogram to 2 milligrams, or 0.1 milligrams to 2 grams; or from 10 milligrams to 1 gram, or from 50 milligrams to 500 milligrams or from 1 microgram to 20 milligrams; or from 1 microgram to 10 milligrams; or from 0.1 milligrams to 2 milligrams.

The pharmaceutical compositions can take any suitable form (e.g, liquids, aerosols, solutions, inhalants, mists, sprays; or solids, powders, ointments, pastes, creams, lotions, gels, patches and the like) for administration by any desired route (e.g, pulmonary, inhalation, intranasal, oral, buccal, sublingual, parenteral, subcutaneous, intravenous, intramuscular, intraperitoneal, intrapleural, intrathecal, transdermal, transmucosal, rectal, and the like). For example, a pharmaceutical composition of the invention may be in the form of an aqueous solution or powder for aerosol administration by inhalation or insufflation (either through the mouth or the nose), in the form of a tablet or capsule for oral administration; in the form of a sterile aqueous solution or dispersion suitable for administration by either direct injection or by addition to sterile infusion fluids for intravenous infusion; or in the form of a lotion, cream, foam, patch, suspension, solution, or suppository for transdermal or transmucosal administration.

A pharmaceutical composition can be in the form of an orally acceptable dosage form including, but not limited to, capsules, tablets, buccal forms, troches, lozenges, and oral liquids in the form of emulsions, aqueous suspensions, dispersions or solutions. Capsules may contain mixtures of a compound of the present invention with inert fillers and/or diluents such as the pharmaceutically acceptable starches (e.g., corn, potato or tapioca starch), sugars, artificial sweetening agents, powdered celluloses, such as crystalline and microcrystalline celluloses, flours, gelatins, gums, etc. In the case of tablets for oral use, carriers which are commonly used include lactose and corn starch. Lubricating agents, such as magnesium stearate, can also be added. For oral administration in a capsule form, useful diluents include lactose and dried corn starch. When aqueous suspensions and/or emulsions are administered orally, the compound of the present invention may be suspended or dissolved in an oily phase is combined with emulsifying and/or suspending agents. If desired, certain sweetening and/or flavoring and/or coloring agents may be added.

A pharmaceutical composition can be in the form of a tablet. The tablet can comprise a unit dosage of a compound of the present invention together with an inert diluent or carrier such as a sugar or sugar alcohol, for example lactose, sucrose, sorbitol or mannitol. The tablet can further comprise a non-sugar derived diluent such as sodium carbonate, calcium phosphate, calcium carbonate, or a cellulose or derivative thereof such as methyl cellulose, ethyl cellulose, hydroxypropyl methyl cellulose, and starches such as corn starch. The tablet can further comprise binding and granulating agents such as polyvinylpyrrolidone, disintegrants (e.g. swellable crosslinked polymers such as crosslinked carboxymethylcellulose), lubricating agents (e.g. stearates), preservatives (e.g. parabens), antioxidants (e.g. BHT), buffering agents (for example phosphate or citrate buffers), and effervescent agents such as citrate/bicarbonate mixtures.

The tablet can be a coated tablet. The coating can be a protective film coating (e.g. a wax or varnish) or a coating designed to control the release of the active agent, for example a delayed release (release of the active after a predetermined lag time following ingestion) or release at a particular location in the gastrointestinal tract. The latter can be achieved, for example, using enteric film coatings such as those sold under the brand name Eudragit®.

Tablet formulations may be made by conventional compression, wet granulation or dry granulation methods and utilize pharmaceutically acceptable diluents, binding agents, lubricants, disintegrants, surface modifying agents (including surfactants), suspending or stabilizing agents, including, but not limited to, magnesium stearate, stearic acid, talc, sodium lauryl sulfate, microcrystalline cellulose, carboxymethylcellulose calcium, polyvinylpyrrolidone, gelatin, alginic acid, acacia gum, xanthan gum, sodium citrate, complex silicates, calcium carbonate, glycine, dextrin, sucrose, sorbitol, dicalcium phosphate, calcium sulfate, lactose, kaolin, mannitol, sodium chloride, talc, dry starches and powdered sugar. Preferred surface modifying agents include nonionic and anionic surface modifying agents. Representative examples of surface modifying agents include, but are not limited to, poloxamer 188, benzalkonium chloride, calcium stearate, cetostearyl alcohol, cetomacrogol emulsifying wax, sorbitan esters, colloidal silicon dioxide, phosphates, sodium dodecylsulfate, magnesium aluminum silicate, and triethanolamine.

A pharmaceutical composition can be in the form of a hard or soft gelatin capsule. In accordance with this formulation, the compound of the present invention may be in a solid, semi-solid, or liquid form.

A pharmaceutical composition can be in the form of a sterile aqueous solution or dispersion suitable for parenteral administration. The term parenteral as used herein includes subcutaneous, intracutaneous, intravenous, intramuscular, intra-articular, intraarterial, intrasynovial, intrasternal, intrathecal, intralesional and intracranial injection or infusion techniques.

A pharmaceutical composition can be in the form of a sterile aqueous solution or dispersion suitable for administration by either direct injection or by addition to sterile infusion fluids for intravenous infusion, and comprises a solvent or dispersion medium containing, water, ethanol, a polyol (e.g., glycerol, propylene glycol and liquid polyethylene glycol), suitable mixtures thereof, or one or more vegetable oils. Solutions or suspensions of the compound of the present invention as a free base or pharmacologically acceptable salt can be prepared in water suitably mixed with a surfactant. Examples of suitable surfactants are given below. Dispersions can also be prepared, for example, in glycerol, liquid polyethylene glycols and mixtures of the same in oils.

The pharmaceutical compositions for use in the methods of the present invention can further comprise one or more additives in addition to any carrier or diluent (such as lactose or mannitol) that is present in the formulation. The one or more additives can comprise or consist of one or more surfactants. Surfactants typically have one or more long aliphatic chains such as fatty acids which enables them to insert directly into the lipid structures of cells to enhance drug penetration and absorption. An empirical parameter commonly used to characterize the relative hydrophilicity and hydrophobicity of surfactants is the hydrophilic-lipophilic balance (“HLB” value). Surfactants with lower HLB values are more hydrophobic, and have greater solubility in oils, while surfactants with higher HLB values are more hydrophilic, and have greater solubility in aqueous solutions. Thus, hydrophilic surfactants are generally considered to be those compounds having an HLB value greater than about 10, and hydrophobic surfactants are generally those having an HLB value less than about 10. However, these HLB values are merely a guide since for many surfactants; the HLB values can differ by as much as about 8 HLB units, depending upon the empirical method chosen to determine the HLB value.

Among the surfactants for use in the compositions of the invention are polyethylene glycol (PEG)-fatty acids and PEG-fatty acid mono and diesters, PEG glycerol esters, alcohol-oil transesterification products, polyglyceryl fatty acids, propylene glycol fatty acid esters, sterol and sterol derivatives, polyethylene glycol sorbitan fatty acid esters, polyethylene glycol alkyl ethers, sugar and its derivatives, polyethylene glycol alkyl phenols, polyoxyethylene-polyoxypropylene (POE-POP) block copolymers, sorbitan fatty acid esters, ionic surfactants, fat-soluble vitamins and their salts, water-soluble vitamins and their amphiphilic derivatives, amino acids and their salts, and organic acids and their esters and anhydrides.

The present invention also provides packaging and kits comprising pharmaceutical compositions for use in the methods of the present invention. The kit can comprise one or more containers selected from the group consisting of a bottle, a vial, an ampoule, a blister pack, and a syringe. The kit can further include one or more of instructions for use in treating and/or preventing a disease, condition or disorder of the present invention, one or more syringes, one or more applicators, or a sterile solution suitable for reconstituting a pharmaceutical composition of the present invention.

All percentages and ratios used herein, unless otherwise indicated, are by weight. Other features and advantages of the present invention are apparent from the different examples. The provided examples illustrate different components and methodology useful in practicing the present invention. The examples do not limit the claimed invention. Based on the present disclosure the skilled artisan can identify and employ other components and methodology useful for practicing the present invention.

### Examples

### Apilimod is a Highly Specific PIKfyve Inhibitor

Protein kinase profiling was conducted to identify cellular kinase targets of apilimod (DiscoveRx, Fremont, Calif.). A dissociation constant (K_{d}) study was performed using apilimod at increasing concentrations (0.05-3000 nM) against PIKfyve, a known target of apilimod. The experiment was performed in duplicate and the K_{d} was determined to be 0.075 nM (range 0.069-0.081 nM).

Next, apilimod was screened against a comprehensive panel of kinases (PIKfyve not included). In total, 456 kinases, including disease-relevant kinases, were assayed for their ability to bind with apilimod. The screening concentration of apilimod was 1 μM, a concentration that is >10,000 times greater than the K_{d} for apilimod against PIKfyve. The results from the screen showed that apilimod did not bind to any of the 456 kinases tested.

### Rationale of Apilimod in Charcot-Marie Tooth Disease

Myotubularin-related proteins (MTMRs) represent a broad family of ubiquitously expressed PTP (protein tyrosine phosphatase)—like phosphatase proteins, which is highly conserved among eukaryotes. The MTMR family is comprised of 14 members in humans, 8 of which are catalytically active proteins, while 6 are catalytically inactive (1). Catalytically active MTMRs are 3-phosphatases acting on both PtdIns3P and PtdIns(3,5)P₂ phosphoinositides (PIs).

Bolino and colleagues first demonstrated that loss of function mutations in the MTMR2 gene causes autosomal recessive demyelinating Charcot-Marie-Tooth type 4B1 (CMT4B1) neuropathy, characterized by childhood onset, proximal and distal muscular weakness and atrophy, sensory deficits, kyphoscoliosis, and cranial nerve involvement (2, 3). MTMR2 is a phospholipid phosphatase acting on PtdIns3P and PtdIns(3,5)P₂ phosphoinositides.

Vaccardi and colleagues have provided evidence that an imbalance of PtdIns(3,5)P₂ in Schwann cells causes myelin outfoldings in MTMR2-null nerves. Genetic and pharmacological inhibition of PIKfyve, the kinase that produces PtdIns(3,5)P₂ from PtdIns3P, rescues myelin outfoldings both in vitro and in vivo (4).

In addition to MTMR2, loss of MTMR13 and MTMR5, both catalytically inactive partners of MTMR2, causes CMT4B2 and CMT4B3 neuropathy, respectively, characterized by clinical features very similar to CMT4B1, although less severe (5).

The hallmark of all CMT4B disorders is the presence of myelin outfoldings in the nerves which consists of redundant loops of myelin around a main myelinated axon.

In accordance with one aspect of the present invention, apilimod acts to correct the imbalance of phosphoinositides and myelin outfolding in CMT4B1 and in CMT4B disorders generally, e.g. CMT4B2(MTMR13) and CMT4B3(MTMR5), and the larger group of CMT type 4 based disorders shown in Table 1.

TABLE 1
CMT Type 4 Neuropathies and Gene

| TYPE | GENE |
|---|---|
| CMT4A | GDAP1 |
| CMT4B1 | MTMR2 |
| CMT4B2 | MTMR13 |
| CMT4B3 | MTMR5 |
| CMT4C | SH3TC2 |
| CMT4D | NDRG1 |
| CMT4E | EGR2 |
| CMT4F | PRX |
| CMT4G | HK1 |
| CMT4H | FDG4 |
| CMT4J | FIG4 |

### REFERENCES

1. Hnia K, Vaccari I, Bolino A, Laporte J. Myotubularin phosphoinositide phosphatases: cellular functions and disease pathophysiology. Trends Mol Med. 2012; 18(6):317-27.
2. Bolino A, Brancolini V, Bono F, Bruni A, Gambardella A, Romeo G, et al. Localization of a gene responsible for autosomal recessive demyelinating neuropathy with focally folded myelin sheaths to chromosome 11q23 by homozygosity mapping and haplotype sharing. Hum Mol Genet. 1996; 5(7):1051-4.
3. Bolino A, Muglia M, Conforti F L, LeGuern E, Salih M A, Georgiou D M, et al. Charcot-Marie-Tooth type 4B is caused by mutations in the gene encoding myotubularin-related protein-2. Nat Genet. 2000; 25(1):17-9.
4. Vaccari I, Dina G, Tronchere H, Kaufman E, Chicanne G, Cerri F, et al. Genetic interaction between MTMR2 and FIG4 phospholipid phosphatases involved in Charcot-Marie-Tooth neuropathies. PLoS Genet. 2011; 7(10):e1002319.
5. Azzedine H, Bolino A, Taieb T, Birouk N, Di Duca M, Bouhouche A, et al. Mutations in MTMR13, a new pseudophosphatase homologue of MTMR2 and Sbf1, in two families with an autosomal recessive demyelinating form of Charcot-Marie-Tooth disease associated with early-onset glaucoma. Am J Hum Genet. 2003; 72(5):1141-53.

1-13. (canceled)

14. A method of reducing PI(3,5)P₂ levels in neuronal cells, the method comprising delivering to the cells apilimod in an amount effective to selectively inhibit PIKfyve activity in the neuronal cells.

15. The method of claim 14, wherein the neuronal cells are in vitro.

16. An method for determining whether apilimod inhibits PIKfyve activity in a neuronal cell, the method comprising contacting the neuronal cell with the apilimod, in vitro, and measuring PI(3,5)P₂ levels in the neuronal cells, wherein a decrease in PI(3,5)P₂ level indicates that the apilimod inhibits PIKfyve activity in the neuronal cell.
